(19) United States
(12) Patent Application Publication
(10) Pub. No.: US 2025/0281719 A1
(43) Pub. Date: Sep. 11, 2025

(54) INTRODUCER SHEATH WITH A STRAIGHT LOADING SHEATH HUB

(71) Applicant: Boston Scientific Scimed, Inc., Maple Grove, MN (US)

(72) Inventors: Judith Tiferes Wang, Shoreview, MN (US); Qian Liu, Plymouth, MN (US)

(73) Assignee: Boston Scientific Scimed, Inc., Maple Grove, MN (US)

(21) Appl. No.: 19/075,045

(22) Filed: Mar. 10, 2025

Related U.S. Application Data
(60) Provisional application No. 63/563,690, filed on Mar. 11, 2024.

(51) Int. Cl. A61M 25/00; A61M 25/06; A61M 39/06

(52) U.S. Cl. CPC A61M 25/0097; A61M 25/0662; A61M 39/06; A61M 2039/0633; A61M 2205/0216

(57) ABSTRACT

An introducer sheath assembly includes an introducer sheath including a hub at the proximal end region of the introducer sheath and an elongate shaft extending distally from the hub. The hub defines a port in fluid communication with a lumen extending through the elongate shaft. A longitudinal port introducer is coupled to and extends proximally from the port of the hub. The longitudinal port introducer includes a housing defining a passageway extending therethrough and a plurality of access ports in the housing, the plurality of access ports including a first access port and a second access port, wherein one of the first and second access ports is axially aligned with the longitudinal axis of the elongate shaft and hub.

### CROSS REFERENCE TO RELATED APPLICATIONS

The present application claims the benefit of U.S. Provisional Patent Application Ser. No. 63/563,690, filed Mar. 11, 2024, the disclosure of which is incorporated herein by reference.

### TECHNICAL FIELD

The present disclosure pertains to introducer sheaths for delivering intravascular medical devices. More specifically, the present disclosure relates to an introducer sheath with a sheath hub configured for receiving a medical device axially through the sheath hub while another medical device, such as a catheter or blood pump, is positioned through the introducer sheath.

### BACKGROUND

In various procedures for delivering intravascular medical devices, a sheath is inserted into a blood vessel of a patient, for example a femoral artery, and one or more medical devices may be advanced through the sheath and into the patient's vasculature. In various instances, the medical devices include catheters or other devices, such as a blood pump. A hub may be incorporated at a proximal end of the sheath to provide access to a lumen of an elongate shaft of the sheath. The hub may include one or more seals, e.g., hemostasis seals, to reduce blood leakage as devices are being inserted, positioned, and removed. In various instances, there may be a desire to fix the position of the medical device within the sheath and vasculature. Additionally, there may be a desire to reposition the medical device within the vasculature. Additionally, there may be a desire to insert several medical devices within the vasculature at the same time. Thus, there is a need for improved introducer sheaths permitting insertion of multiple medical devices through the introducer sheath at the same time and including sealing mechanisms to reduce blood leakage past the medical device(s) and/or tightening mechanisms for securing the medical device(s), such as a catheter or blood pump, within the sheath and vasculature that also allow insertion of one or more additional medical devices through the introducer sheath and/or repositioning of the medical device in the vasculature.

### BRIEF SUMMARY

This disclosure provides design, material, manufacturing method, and use alternatives for medical devices, including introducer and/or repositioning sheaths.

A first example is an introducer sheath assembly. The introducer sheath assembly includes an introducer sheath. The introducer sheath includes a hub at a proximal end region of the introducer sheath. The hub defines a port. An elongate shaft extends distally from the hub to a distal end region of the introducer sheath. The elongate shaft defines a lumen extending therethrough. A longitudinal port introducer extends proximally from the port of the hub. The longitudinal port introducer includes a housing defining a passageway extending therethrough and a plurality of access ports in the housing. The plurality of access ports include a first access port and a second access port. The first access port extends at an oblique angle from the second access port.

Alternatively or additionally to any of the examples herein, in another example, the longitudinal port introducer is integral with the hub.

Alternatively or additionally to any of the examples herein, in another example, the longitudinal port introducer is removably coupled to the hub.

Alternatively or additionally to any of the examples herein, in another example, the angle is an acute angle.

Alternatively or additionally to any of the examples herein, in another example, the first access port extends through a side surface between the proximal end and the distal end of the longitudinal port introducer.

Alternatively or additionally to any of the examples herein, in another example, the first access port, the second access port, or both, further comprises a tightening port.

Alternatively or additionally to any of the examples herein, in another example, the second access port extends through a proximal end of the housing of the longitudinal port introducer.

Alternatively or additionally to any of the examples herein, in another example, the plurality of access ports are each configured to receive a separate medical device and provide the respective medical device access to the lumen of the elongate shaft of the introducer sheath.

Alternatively or additionally to any of the examples herein, in another example, at least one of the access ports in the longitudinal port introducer includes an elastomeric seal.

Alternatively or additionally to any of the examples herein, in another example, the elastomeric seal is configured to seal the second access port.

Alternatively or additionally to any of the examples herein, in another example, the elastomeric seal is included in a plurality of elastomeric seals that include a first elastomeric seal and a second elastomeric seal, wherein the housing further comprises a third access port, and wherein the first elastomeric seal is configured to seal the second access port and the second elastomeric seal is configured to seal the third access port.

Alternatively or additionally to any of the examples herein, in another example, the hub includes a hemostasis valve.

Alternatively or additionally to any of the examples herein, in another example, the longitudinal port introducer includes a seal displacement mechanism.

Alternatively or additionally to any of the examples herein, in another example, the seal displacement mechanism includes one or more protrusions extending distally from the housing of the longitudinal port introducer configured to engage the hemostasis valve.

Alternatively or additionally to any of the examples herein, in another example, the one or more protrusions is configured to open the hemostasis valve when the longitudinal port introducer is coupled to the hub.

Another example is an introducer sheath assembly. The introducer assembly includes an introducer sheath including a hub at a proximal end of the introducer sheath. The hub defines a port including a hemostasis valve therein. An elongate shaft extends distally from the hub to a distal end of the introducer sheath. The elongate shaft defines a lumen extending therethrough. A longitudinal port introducer is detachably coupled to the port of the hub. The longitudinal port introducer includes a housing defining a passageway extending therethrough and a plurality of access ports in the housing. The plurality of access ports includes a first access port and a second access port. The second access port is axially aligned with the port of the hub and the first access port extends at an angle from the second access port. Each of the first and second access ports is configured to receive a separate medical device therethrough and provide the respective medical device access to the port of the hub for advancement into the lumen of the elongate shaft of the introducer sheath.

Alternatively or additionally to any of the examples herein, in another example, the longitudinal port introducer includes a seal displacement mechanism extending distally from the housing of the longitudinal port introducer, wherein the seal disengagement mechanism is configured to engage the hemostasis valve when the longitudinal port introducer is coupled to the port of the hub.

Another example is a method of advancing a plurality of medical devices through an introducer sheath. The method includes advancing a first medical device through a port of a hub of the introducer sheath in an axial direction extending along a longitudinal axis of the introducer sheath, and advancing a second medical device through a second access port of a longitudinal port introducer coupled to the hub of the introducer sheath and through the port of the hub of the introducer sheath in an axial direction extending along the longitudinal axis of the introducer sheath while a proximal end region of the first medical device extends proximally through a first access port of the longitudinal port introducer at an angle to the second access port.

Alternatively or additionally to any of the examples herein, in another example, the method includes coupling the longitudinal port introducer to the hub of the introducer sheath after advancing the first medical device through the port of the hub of the introducer sheath.

Alternatively or additionally to any of the examples herein, in another example, the longitudinal port introducer is pre-loaded onto the first medical device prior to advancing the first medical device through the port of the hub of the introducer sheath.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a side view of an example introducer sheath extending into a blood vessel;

FIG. 2 is a cross-sectional view of a portion of the example introducer sheath of FIG. 1 inserted into a blood vessel, and a medical device inserted into the introducer sheath;

FIG. 3 is a perspective view of the example introducer sheath of FIG. 1;

FIGS. 4A-4C are cross-sectional views of variations of a longitudinal port introducer;

FIG. 5 is a view of an elastomeric seal in an access port of an example longitudinal port introducer;

FIG. 6 is a cross-sectional view of a portion of the introducer sheath of FIG. 3;

FIG. 7 is a cross-sectional view of the introducer sheath of FIG. 3 with a plurality of medical devices passing through the hemostasis valve; and

FIGS. 8A-8C illustrate a series of steps of inserting and advancing a plurality of medical devices through the introducer sheath.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. For instance, various elements of the Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Hemostasis valve hub assemblies provided with an introducer sheath facilitate insertion and positioning of one or more medical devices (e.g., catheters, blood pumps, guidewires, etc.) through the introducer sheath while also helping to prevent blood from leaking during a medical procedure. Some embodiments of the present disclosure feature assemblies with components that permit insertion of and/or positioning of multiple medical devices though the introducer sheath at the same time. That is, a plurality of medical devices such as a catheter and/or guidewire and a blood pump can be inserted and/or positioned through the introducer sheath at the same time. For instance, each of the medical devices can each be inserted via a port in a substantially longitudinal manner. Inserting each of the medical devices substantially longitudinally (e.g., straight into a lumen of the introducer sheath) may reduce any mechanical stress (e.g., angular stress) and/or may mitigate any alignment issues encountered during insertion and/or use of the medical devices. For instance, a first medical device (e.g., a blood pump) may be inserted longitudinally via a single lumen of the introducer sheath and a second medical device may be inserted longitudinally via the same single lumen of the introducer sheath substantially in parallel with the first medical device while the first medical device is still inserted through the introducer sheath.

For example, a first medical device (e.g., a blood pump) may be inserted into a port of an introducer hub of the introducer sheath and may be locked in a given configuration (e.g., locked proximate to a side surface of a component), such as with a Tuohy Borst valve. A second medical device (e.g., a catheter, a balloon, a guidewire, a stent and/or other type of percutaneous coronary intervention (PCI) tool) may be subsequently inserted into the same port of the introducer hub. Such insertion and locking of the first medical device in a single port may ease insertion and/or delivery of the second medical device via the same single port, as detailed herein. Further, some embodiments may also provide components that facilitate convenient repositioning of the one or more medical devices relative to the introducer sheath.

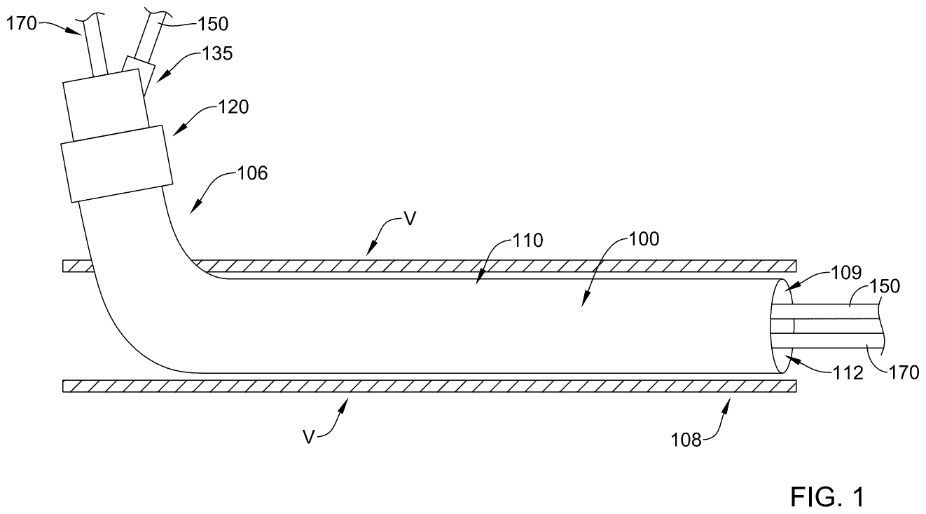

FIG. 1 illustrates a side view of an introducer sheath 100 inserted at least partially into a blood vessel V, shown in cross-section. While the disclosure herein is made with reference largely to the introducer sheath 100, and components thereof, the disclosure may also apply to a repositioning sheath, as will be discussed further herein. Further, it is noted that various elements may be added or omitted from the introducer sheathes and/or the valve hubs described herein that depict illustrative embodiments and are not intended to limit the scope of the disclosure.

In some embodiments, the introducer sheath 100 may be used for facilitating the passage of various medical devices, such as a catheter, a guidewire, or a blood pump as will be described further herein, through the introducer sheath 100 and into the blood vessel V. Hence, in some instances the introducer sheath 100 may be referred to as a large bore introducer sheath. The introducer sheath 100 includes a proximal end region 106 proximate a proximal end of the introducer sheath 100 and a distal end region 108 proximate a distal end of the introducer sheath 100 that is opposite the proximal end region 106. A body portion 110 (e.g., elongate shaft) of the introducer sheath 100 extends between the proximal end region 106 and the distal end region 108, and the body portion 110 defines a lumen 112 of the introducer sheath 100. The introducer sheath 100 includes a proximal opening (not shown) adjacent the proximal end region 106 and a distal opening 109 adjacent the distal end region 108, with the lumen 112 extending from the proximal opening to the distal opening 109. The introducer sheath 100, or components thereof, may be formed by various materials, such as polymeric and/or metallic materials. In some instances, the introducer sheath 100, such as the elongate shaft of the introducer sheath 100, may include an additional surface coating, such as but not limited to, silicone, PET, or other applicable polymer.

A hub 120 may be provided at the proximal end region 106 to provide access to the lumen 112 of the introducer sheath 100. In some instances, the hub 120 may be configured for hemostasis by, for example, helping to prevent blood from leaking out of the introducer sheath 100 during use. In such instances, the hub 120 may include one or more seals configured to seal the lumen 112. In other instances, the hub 120 may be devoid of any seals and/or valves.

The introducer sheath assembly, including the introducer sheath 100, may include a longitudinal port introducer 135, as detailed herein. The longitudinal port introducer 135 may be provided with or attached to the introducer sheath 100, as detailed herein. That is, longitudinal port introducer 135 may be an integral component on the introducer sheath 100 or may be a separate component that is configured to couple (e.g., removably couple) to the introducer sheath 100. The introducer sheath 100 may be configured to permit a plurality of medical devices to be longitudinally inserted along a longitudinal axis of the introducer sheath 100.

For example, a plurality of medical devices such as a blood pump 150 and an ancillary medical device 170 (e.g., catheter, guidewire, etc.), may be inserted and/or positioned concurrently through the hub 120 and lumen 112 of the introducer sheath 100 and into the blood vessel V, and the hub 120 may maintain hemostasis between the medical devices 150, 170, the introducer sheath 100, and the external surroundings. After insertion of the medical devices, fixation of the axial and/or radial position of one or more of the medical devices (e.g., the blood pump 150) may be desired to ensure that the one or more medical devices (and any device coupled thereto) is maintained in a proper position during use. It may also be desired for the medical personnel to reposition the medical device after insertion. As such, the hub 120 and/or the longitudinal port introducer 135 may include a tightening port (e.g., tightening port 147, as described herein), provided with or attachable thereto, that provides for the fixation of the medical device with respect to the hub 120, the longitudinal port introducer 135, and blood vessel V, as will be described further herein. A tightening port (e.g., the tightening port 147 as described herein) may allow for the repositioning of the medical device with respect to the hub 120, the longitudinal port introducer 135, and the blood vessel V.

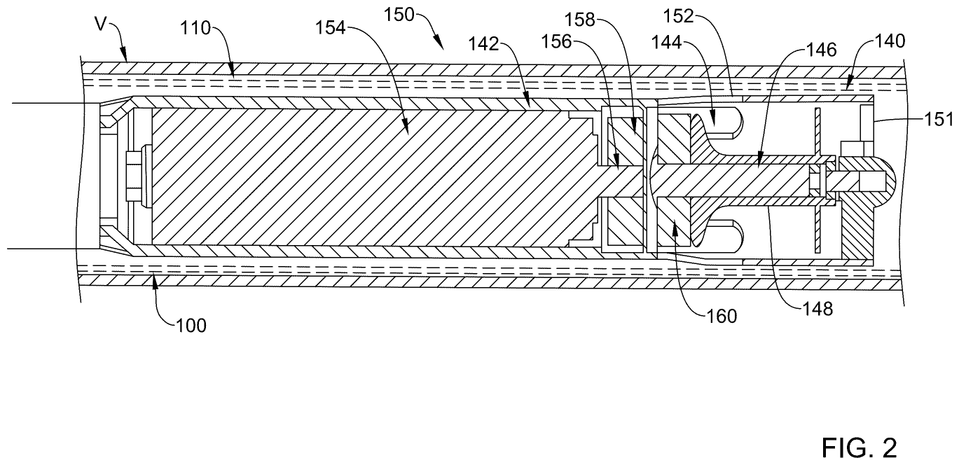

FIG. 2 illustrates a cross-sectional view of the body portion 110 of the introducer sheath 100 of FIG. 1 upon insertion of a medical device, illustratively a blood pump 150, into the introducer sheath 100. As noted above, the medical devices of FIG. 1 may be coupled to or include the blood pump 150, with the medical device extending outside the blood vessel V and the introducer sheath 100. The blood pump 150 may be advanced through the blood vessel V and positioned in a target location, such as a target cardiac location (e.g., the left ventricle), via the introducer sheath 100. The blood pump 150 may generally include an impeller assembly housing 140 and a motor housing 142. In some embodiments, the impeller assembly housing 140 and the motor housing 142 may be integrally or monolithically constructed. In other instances, the impeller assembly housing 140 and the motor housing 142 may be separate components. The impeller assembly housing 140 carries an impeller assembly 144 therein. The impeller assembly 144 may include an impeller shaft 146 and an impeller 148 that rotate relative to the impeller assembly housing 140 to drive blood through the blood pump 150. More specifically, the rotation of the impeller 148 causes blood to flow from a blood inlet 151 formed on the impeller assembly housing 140, through the impeller assembly housing 140, and out of a blood outlet 152 formed on the impeller assembly housing 140. In some embodiments, the impeller shaft 146 and the impeller 148 may be integrally formed, whereas, in other embodiments the impeller shaft 146 and the impeller 148 may be separate components. As shown in FIG. 2, the inlet 151 may be formed on an end portion of the impeller assembly housing 140 and the outlet 152 may be formed on a side portion of the impeller assembly housing 140. In other embodiments, the inlet 151 and/or the outlet 152 may be formed on other portions of the impeller assembly housing 140. In some embodiments, the impeller assembly housing 140 may be coupled to a distally extending cannula, and the cannula may receive and deliver blood to the inlet 151.

With continued reference to FIG. 2, the motor housing 142 carries a motor 154, and the motor 154 is configured to rotatably drive the impeller 148 relative to the impeller assembly housing 140. In the illustrated embodiment, the motor 154 rotates a drive shaft 156, which is coupled to a driving magnet 158. Rotation of the driving magnet 158 causes rotation of a driven magnet 160, which is connected to the impeller assembly 144. More specifically, in embodiments incorporating the impeller shaft 146, the impeller shaft 146 and the impeller 148 are configured to rotate with the driven magnet 160. In other embodiments, the motor 154 may be coupled to the impeller assembly 144 via other components. While the introducer sheath 100 is illustrated above with the use of the blood pump 150 and the ancillary medical device 170, various other medical devices may be used in conjunction with the introducer sheath 100 and the hemostasis valve hub 120.

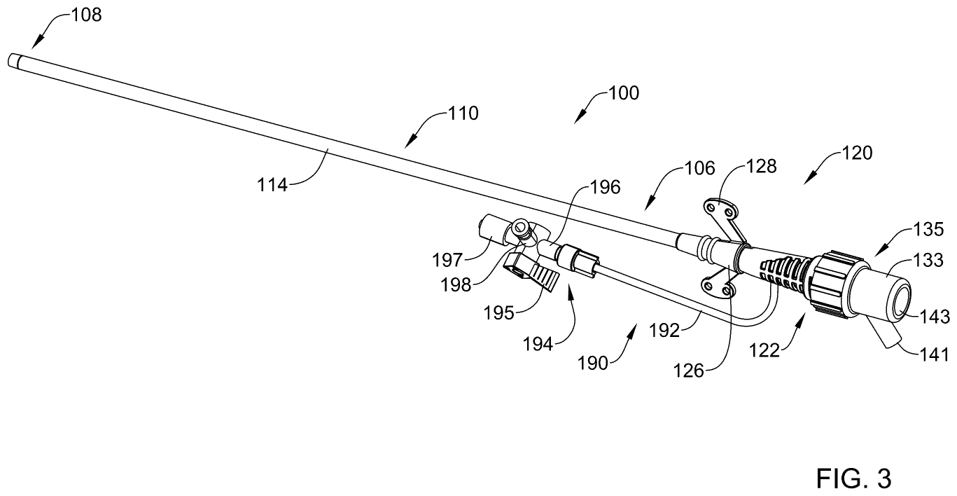

FIG. 3 is a perspective view of the introducer sheath assembly of FIG. 1 in which further details of the introducer sheath 100 are illustrated. For example, the introducer sheath 100 includes the longitudinal port introducer 135, the hub 120, and an elongate shaft 114 extending distally from the hub 120. The elongate shaft 114 may define the body portion 110 of the sheath 100.

The sheath 100 may also include a flush line 190 extending from the hub 120. The flush line 190 may include a tubular member 192 extending from the hub 120 and in fluid communication with a lumen of the hub 120. The tubular member 192 may extend to a stopcock 194, such as a three-way stopcock. The stopcock 194 may include a first leg 196 coupled to the tubular member 192, a second leg 197, a third leg 198, and a lever 195 that is rotatable to selectively open/close fluid access between the first, second and/or third legs 196, 197, 198. Each of the legs 196, 197, 198 may include a connector, such as a luer connector, as desired.

The hub 120 may also include a strain relief 126 configured to provide a transition flexibility along the proximal end region 106. The strain relief 126 may include a body attached to a main body of the hub 120, as will be described further herein. The strain relief 126 may include one or more suture pads 128 extending outward therefrom. For example, the strain relief 126 may include first and second suture pads 128 extending from opposite sides of the strain relief 126. The suture pads 128 may facilitate securing the hub 120 against the patient once the introducer sheath 100 has been positioned in the blood vessel of the patient. For example, each suture pad 128 may include at least one opening extending therethrough for receiving a suture used to suture the hub 120 to the skin of the patient.

The suture pads 128 may be angled at an acute angle relative to the longitudinal axis of the sheath 100. For instance, the suture pads 128 may be angled toward the distal end region 108 of the sheath 100 such that the free ends of the suture pads 128 are positioned distal of the base ends of the suture pads 128. In some instances, the angle between the suture pads 128 and the longitudinal axis of the sheath 100 may be about 30 degrees to about 90 degrees, about 35 degrees to about 85 degrees, about 40 degrees to about 80 degrees, about 45 degrees to about 75 degrees, about 50 degree to about 70 degrees, or about 55 degrees to about 65 degrees. In some instances, the angle between the suture pads 128 and the longitudinal axis of the sheath 100 may be about 35 degrees, 40 degrees, 45 degrees, 50 degrees, 55 degrees, or 60 degrees.

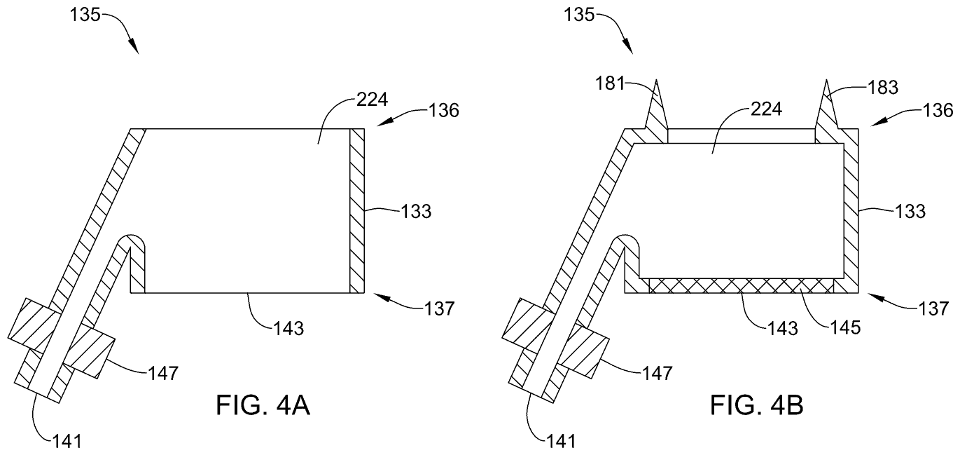

The longitudinal port introducer 135 may be coupled (e.g., releasably coupled) to the hub 120 such that the lumen through the longitudinal port introducer 135 may be in communication with the port 122 of the hub 120 leading into the lumen 112 of the elongate shaft 114 of the introducer sheath 100. As illustrated in FIG. 3, the longitudinal port introducer 135 may extend proximally from the port 122 of the valve hub 120. The longitudinal port introducer 135 may include a housing 133. The housing 133 may extend substantially proximally from the port 122. Referring to FIGS. 4A-4C, the housing 133 of the longitudinal port introducer 135 may define a passageway 224 extending therethrough (e.g., extending from the distal end 136 of the housing 133 to the proximal end 137 of the housing 133). The passageway 134 may be in fluid communication with the passageway of the port 122 when coupled thereto. The passageway 134 may be in fluid communication with a plurality of access ports of the longitudinal port introducer 135, providing access for multiple medical devices to be inserted therein. For instance, the housing 133 may define a plurality of access ports including a first access port 141 and a second access port 143, where the first access port 141 and the second access port 143 are each in fluid communication with the passageway 134. Having each of the access ports be in fluid communication with the passageway 134 and the passageway of the port 222 of the hub 120 of the introducer sheath 100 may promote aspects herein such as permitting insertion of a plurality of medical devices through a single port 122 via the longitudinal port introducer 135. Notably, the single port 122 along with the access ports 141, 143 may each be configured in such a manner to thereby permit the insertion of a plurality of medical devices in a longitudinal manner through the single port 122 and along the longitudinal axis of the elongate shaft 114 of the introducer sheath 100, unlike other approaches such as those which may employ a plurality of ports in an introducer sheath itself to permit insertion of a medical device at an acute angle to the longitudinal axis of the introducer sheath. As such, the approaches herein may permit a plurality of medical devices to be inserted into the lumen of the elongate shaft 114 of the introducer sheath 100 via the port 122 in a straight or relatively straight manner with respect to a longitudinal axis of the introducer sheath 100 (e.g., substantially parallel with the longitudinal axis of the elongate shaft 114 of the introducer sheath 100), unlike other approaches such as those which insert a plurality of medical devices in a plurality of ports of the insertion sheath.

In some embodiments, the first access port 141 may be at an angle, e.g., an acute angle, to the second access port 143. Having the first access port 141 be located at an angle to the second access port 143 may promote aspects herein such as permitting a plurality of medical devices to be inserted via the port 122 of the introducer sheath 100 in a straight or substantially straight manner with respect to a longitudinal axis of the introducer sheath 100. For instance, in some embodiments the first access port 141 may be at an acute angle to the second access port 143. In some instances, the second access port 143 may be aligned with the longitudinal axis of the introducer sheath 100 and thus may extend along a longitudinal axis (e.g., coaxial with the longitudinal axis, or otherwise be at a zero-degree angle relative to) the longitudinal axis of the introducer sheath 100. In some instances, the angle between the first access port 141 and the second access port 143 may be in a range from about 5 degrees to about 85 degrees. For instance, the angle between the first access port 141 and the second access port 143 may be a value in a range from about 10 degrees to about 75 degrees, about 10 degrees to about 45 degrees, about 15 degrees to about 60 degrees, about 45 degrees to about 60 degrees, or about 15 degrees to about 75 degrees in some instances. In some instances, the angle between the first access port 141 and the second access port 143 may be about 10 degrees, about 15 degrees, about 20 degrees, about 25 degrees, about 30 degrees, 35 degrees, 40 degrees, 45 degrees, 50 degrees, 55 degrees, or 60 degrees. In some instances, the angle between the longitudinal axis of the first access port 141 and the longitudinal axis of the second access port 143 may be an acute angle. For instance, the second access port 143 may extend along the longitudinal axis of the introducer sheath 100 (e.g., coaxial with the longitudinal axis, or otherwise be at a zero-degree angle with respect to the longitudinal axis of the introducer sheath 100) and the first access port 141 may be at an acute angle with respect to the second access port 143. Having the second access port 143 extend along or parallel to the longitudinal axis of the introducer sheath 100 and having the first access port 141 at an acute angle with respect to the second access port 143 may promote aspects herein such as permitting a plurality of medical devices to be inserted via the port 122 of the hub 120 of the introducer sheath 100 in a straight or relatively straight manner with respect to a longitudinal axis of the introducer sheath 100, unlike other approaches such as those which insert a plurality of medical devices in a plurality of ports of the insertion sheath.

In some embodiments, the first access port 141 may be located in and/or extend from a side surface of the longitudinal port introducer 135. The side surface refers to a surface between the distal end 136 and the proximal end 137 of the longitudinal port introducer 135. For instance, the first access port 141 may be located in a side surface of the longitudinal port introducer 135. For example, the first access port 141 may be located in a side surface and the second access port 143 may be located in the proximal end 137 of the longitudinal port introducer 135. Having the first access port 141 be located in (e.g., extending through) a side surface and the second access port 143 located in (e.g., extending through) the proximal end 137 of the longitudinal port introducer 135 may promote aspects herein such as permitting a plurality of medical devices to be inserted via the port 122 of the hub 120 in a straight or relatively straight manner with respect to a longitudinal axis of the introducer sheath 100, unlike other approaches such as those which insert a plurality of medical devices in a plurality of ports of the insertion sheath. That is, each of the plurality of access ports in the longitudinal port introducer 135 may be configured to receive a respective medical device therein and provide the medical device with access to the body lumen via extending through the body portion of the introducer sheath 100.

In some embodiments, the longitudinal port introducer 135 includes an access port that is configured as a tightening port, e.g., a Tuohy Borst seal. As mentioned, a tightening port includes components that permit and/or provides for the fixation of the medical device with respect to the hub 120, the longitudinal port introducer 135, and blood vessel V, as will be described further herein. The tightening port may also function as a seal to maintain hemostasis (e.g., when a medical device is inserted in the tightening port). The first access port 141, the second access port 143, or both, may be a tightening port. For example, as illustrated in FIGS. 4A-4C, the first access port 141 may be a tightening port 147 including a sealing structure configured to form a hemostasis seal around the medical device inserted therethrough. Having at least the first access port 141 be a tightening port may promote aspects herein such as permitting the selective fixation of a first medical device (e.g., a blood pump) inserted via the first access port 141 which may ease insertion, steering, and/or placement of a second medical device that is inserted via the second access port 143 while the first medical device (e.g., blood pump 150) remains inserted (e.g., and fixed in a given position) via the first access port 141.

In some embodiments, some or all of the access ports of the longitudinal port introducer 135 may be seal-free. Stated differently, the longitudinal port introducer 135 may be without a seal such as an elastomeric seal. In such embodiments, the longitudinal port introducer 135 may be configured with access ports that utilize a seal (e.g., elastomeric seal 260) included in the introducer sheath 100 (rather than a seal in the longitudinal port introducer 135) to maintain hemostasis prior to, during, and after insertion of medical devices via the longitudinal port introducer 135 into the introducer sheath 100. FIG. 4A is a view of an example longitudinal port introducer 135 that includes a second access port 143 that is seal-free. Having some or all of the access ports 141, 143 of the longitudinal port introducer 135 be seal-free may promote aspects herein such as promoting ease of insertion and/or steering of medical devices in the longitudinal port introducer 135 and/or in the introducer sheath 100.

However, in some embodiments the longitudinal port introducer 135 may include a seal such as an elastomeric seal. An elastomeric seal may be disposed at some or all of the access ports 141, 143 in the longitudinal port introducer 135. For instance, FIG. 4B is a view of another example longitudinal port introducer 135 including an elastomeric seal 145 positioned at the second access port 143. The elastomeric seal 145 may be configured to seal the second access port 143. The elastomeric seal 145 may be an elastomeric seal such as a slit valve (e.g., a cross-slit valve), a dome valve, a duckbill valve, or any other desired valve configured to seal around an elongate shaft of a medical device when passed therethrough. In some instances, the elastomeric seal 145 may include one or more slits (e.g., crossing slits) extending entirely through the seal wall and/or one or more slits (e.g., crossing slits) extending only partially through the seal wall. For instance, the elastomeric seal 145 may be a cross-slit valve having a first slit extending into the wall of the seal from a first side of the seal but not extend entirely through the wall of the seal and a second slit extending into the wall of the seal from a second, opposite side of the seal but not extend entirely through the wall of the seal. The first slit may intersect the second slit within the wall of the valve. In some instances, the first slit may be arranged perpendicular to the second slit. The elastomeric seal 145 may be formed of any desired flexible material, such as silicone, polyurethane, etc.

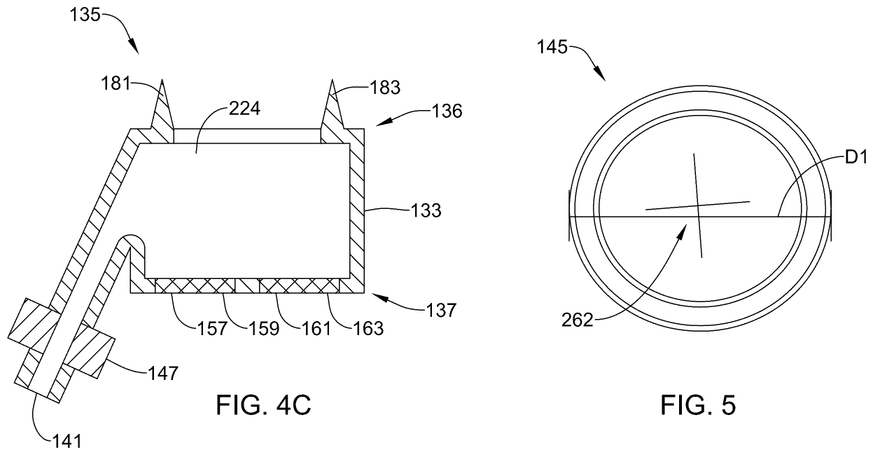

In some embodiments, the longitudinal port introducer 135 may include a seal displacement mechanism. The displacement mechanism may be manifested as one or more protrusions that extends distally from the housing 133 of the longitudinal port introducer 135. For instance, the displacement mechanism may be manifested as a plurality of protrusions 181, 183 extending distally from the housing 133 of the longitudinal port introducer 135, as illustrated in FIG. 5C, for engagement with the seal in the hub 120 of the introducer sheath 100. In other instances, the displacement mechanism may be a protrusion, such as an annular ring or collar, extending distally from the housing 133 for engagement with the seal in the hub 120 of the introducer sheath 100. Employing a seal displacement mechanism such as the plurality of protrusions 181, 183 extending distally from the housing 133 may promote aspects herein such as promoting uniform access through a seal in the introducer sheath 100. That is, the seal displacement mechanism may be configured to provide access to a lumen of the introducer sheath 100 by opening a seal of the introducer sheath 100 and thereby ease insertion, steering, and/or placement of medical devices inserted via the access ports (e.g., access ports 141, 143) of the longitudinal port introducer 135. Yet, at least due to the presence of the elastomeric seal 145 hemostasis can be maintained within the introducer sheath 100 and the longitudinal port introducer 135. For instance, the elastomeric seal 145 may be located proximal to the seal displacement mechanism (e.g., the plurality of protrusions 181, 183) and thereby may ensure that hemostasis can be maintained within the introducer sheath 100 and the longitudinal port introducer 135.

FIG. 4C is a view of an access port of an example longitudinal port introducer 135 including a plurality of elastomeric seals. The plurality of elastomeric seals may include a first elastomeric seal 159 and a second elastomeric seal 163. As illustrated in FIG. 4C, the first elastomeric seal 159 may be positioned in and be configured to seal the second access port 157 and the second elastomeric seal 163 may be positioned at and be configured to seal the third access port 161. The second access port 157 and the third access port 161 may each be located on (e.g., extending through) a proximal end of the longitudinal port introducer 135, as illustrated in FIG. 4C.

The first elastomeric seal 159 and the second elastomeric seal 163 may each be a slit valve (e.g., a cross-slit valve), a dome valve, a duckbill valve, or any other desired valve configured to seal around an elongate shaft of a medical device when passed therethrough. In some instances, the first elastomeric seal 159 and the second elastomeric seal 163 may each include one or more slits (e.g., crossing slits) extending entirely through the seal wall and/or one or more slits (e.g., crossing slits) extending only partially through the seal wall. For instance, the first elastomeric seal 159 and the second elastomeric seal 163 may each be a cross-slit valve having a first slit extending into the wall of the seal from a first side of the seal but not extend entirely through the wall of the seal and a second slit extending into the wall of the seal from a second, opposite side of the seal but not extend entirely through the wall of the seal. The first slit may intersect the second slit within the wall of the valve. In some instances, the first slit may be arranged perpendicular to the second slit. The first elastomeric seal 159 and the second elastomeric seal 163 may each be formed of any desired flexible material, such as silicone, polyurethane, etc. In some embodiments, the first elastomeric seal 159 and the second elastomeric seal 163 may each be the same type of elastomeric seal. Having the first elastomeric seal 159 be configured to seal the second access port 157 and the second elastomeric seal 163 be configured to seal the third access port 161 may promote an aspect described herein such as permitting a plurality of medical devices to be inserted in a straight or substantially straight manner with respect to the longitudinal axis of the introducer sheath 100, for instance, while the first medical device remains inserted (e.g., and fixed in a given position) via the first access port 141.

FIG. 5 is a view of an elastomeric seal 145 in an access port of an example longitudinal port introducer 135. The elastomeric seal 145 may be analogous or similar to other elastomeric seals 159 and/or 163, as described herein. As illustrated in FIG. 5, the seal 145 may be generally cylindrical in shape and may have a circular cross section with partial cross slits 262 arranged within a center of the elastomeric seal 145. The partial cross slits 262 may be configured such that the at least one medical device inserted through the cross slits 262 of the elastomeric seal 145 in the access port, such that the cross-slits 262 sealingly engage with the medical device. The elastomeric seal 145 may be composed of silicone, however various other materials may be used. For example, polymer materials, including elastomeric polymers, thermoset rubber, thermoset elastomer (TSE), or silicone rubber may be used. The elastomeric seal 145 may have a diameter D1 that has a value between 2 mm and 12 mm, for example. The configuration of the elastomeric seal 145 may provide the additional hemostasis advantage for longer duration of usage. The partial cross slit elastomeric seal 145 grips the medical device passed therethrough without absorbing the fluid/blood that may be passing though the device. The configuration of the elastomeric seal 145 may allow the hemostasis of blood for longer duration during usage of the introducer sheath 100 in the blood vessel V. While illustrated as a cross-slit, the seal 145 may have other configurations such as those described herein.

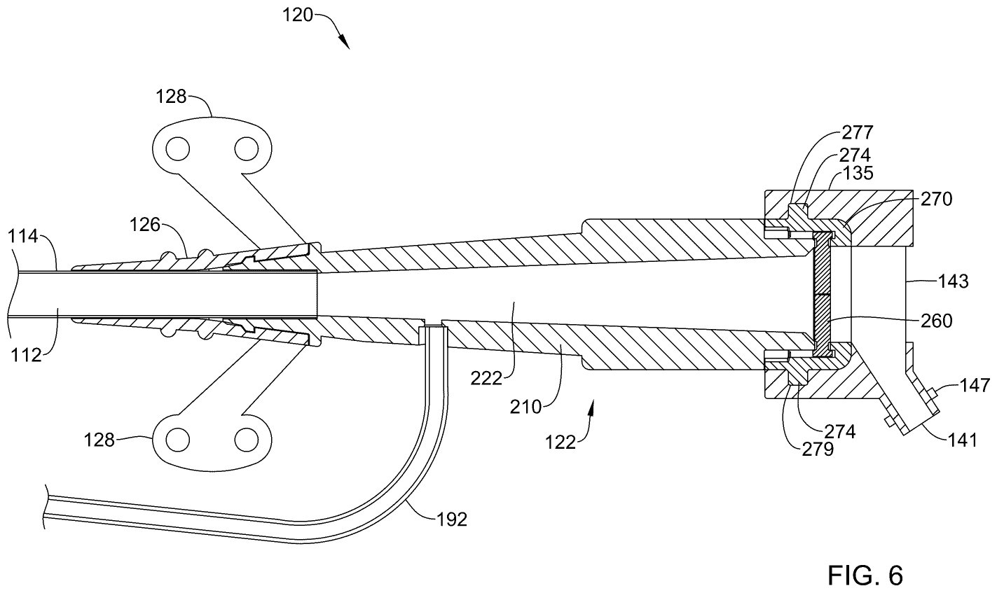

Further details of the components of the hub 120 and the longitudinal port introducer 135 are shown in the cross-sectional view of FIG. 6. The hub 120 may include a port 122 as previously described. The port 122 may be aligned with a longitudinal axis of the sheath 100. The port 122 of hub 120 may include a hub body 210. In some instances, the hub body 210 may be a molded, one-piece structure. For example, the hub body 210 may be molded from a polymeric material. In other instances, the hub body 210 may be formed of two or more components attached together.

The port 122, may include a passageway 222 extending therethrough. For example, the hub body 210 may define the passageway 222. The passageway 222 may be aligned with the lumen 112 of the elongate shaft 114 such that the central longitudinal axis of the lumen 112 of the elongate shaft 114 passes through the passageway 222. For example, the passageway 222 may be co-axial with the lumen 112 of the elongate shaft 114, in some instances. A passageway 224 of the longitudinal port introducer 135 may be in fluid communication with the passageway 222. The passageway 222 and/or the passageway 224 may be in fluid communication with the lumen 112 of the elongate shaft 114 of the introducer sheath 100 extending to the distal opening 109 (See FIG. 1).

In some instances, the port 122 may include a hemostasis seal for preventing blood from egressing from the port 122 during use. For example, the port 122 may include an elastomeric seal 260. The elastomeric seal 260 may be housed in the port 122. For instance, the port lid 270 may be configured such that the elastomeric seal 260 may be captured between an end surface of the main body 210 and an internal surface of a port lid 270, or coupled or secured to the main body 210. The port lid 270 may extend distally beyond the elastomeric seal 260 to define an opening of the port 122 providing access to the passageway 222 of the port 122. In some instances, the port 122 may be a tightening port, as discussed above.

The elastomeric seal 260 may be a slit valve (e.g., a cross-slit valve), a dome valve, a duckbill valve, or any other desired valve configured to seal around an elongate shaft of a medical device when passed therethrough. In some instances, the elastomeric seal 260 may include one or more slits (e.g., crossing slits) extending entirely through the seal wall and/or one or more slits (e.g., crossing slits) extending only partially through the seal wall. For instance, the elastomeric seal 260 may be a cross-slit valve having a first slit extending into the wall of the seal from a first side of the seal but not extend entirely through the wall of the seal and a second slit extending into the wall of the seal from a second, opposite side of the seal but not extend entirely through the wall of the seal. The first slit may intersect the second slit within the wall of the valve. In some instances, the first slit may be arranged perpendicular to the second slit. The elastomeric seal 260 may be formed of any desired flexible material, such as silicone, polyurethane, etc.

As mentioned, the longitudinal port introducer 135 may be integral with or may be coupled to the introducer sheath 100. For instance, the longitudinal port introducer 135 may be removably coupled to a proximal end of the hub 120 of the introducer sheath 100. For instance, a coupler may be provided to removably couple the longitudinal port introducer 135 to the proximal end of the hub 120. For example, the longitudinal port introducer 135 may include a coupling mechanism configured to mate with a coupling mechanism of the hub 120. The coupling mechanism may include snap-fit mechanisms, pogo-pins, threads and corresponding threaded members, and/or other types of coupling mechanisms that friction fit or otherwise mechanically couple (e.g., removably couple) the longitudinal port introducer 135 to the hub 120 of the introducer sheath 100. For example, the longitudinal port introducer 135 may include coupling mechanisms 277 and 279 (e.g., shown as recesses, slots, and/or channels) that are configured to couple to the plurality of locking features 274, such as pins shown in FIG. 6, extending from the hub 120 (e.g., the port lid 270) that may be configured to permit the longitudinal port introducer 135 to be detachably coupled to the hub 120. Yet, in some instances, the coupling mechanisms 277/279 may be manifested as a threaded region, such as an internally threaded region, configured to threadably engage with a threaded region, such as an externally threaded region at a proximal end of the hub 120 (e.g., a threaded region on the hub body 210 or port lid 270). That is, in some embodiments, the longitudinal port introducer 135 may be removably coupled to a proximal end of the hub body 210, the port lid 270, or other component of the hub 120.

However, in some instances, the longitudinal port introducer 135 may be an integral component (e.g., originally manufactured as a non-removable component) of the hub 120 of the introducer sheath 100. In any case, the longitudinal port introducer 135 may be configured to couple to an existing introducer sheath 100 and thereby readily permit a plurality of medical devices to be inserted into and remain in a single port (e.g., port 122) of the introducer sheath 100, as described herein.

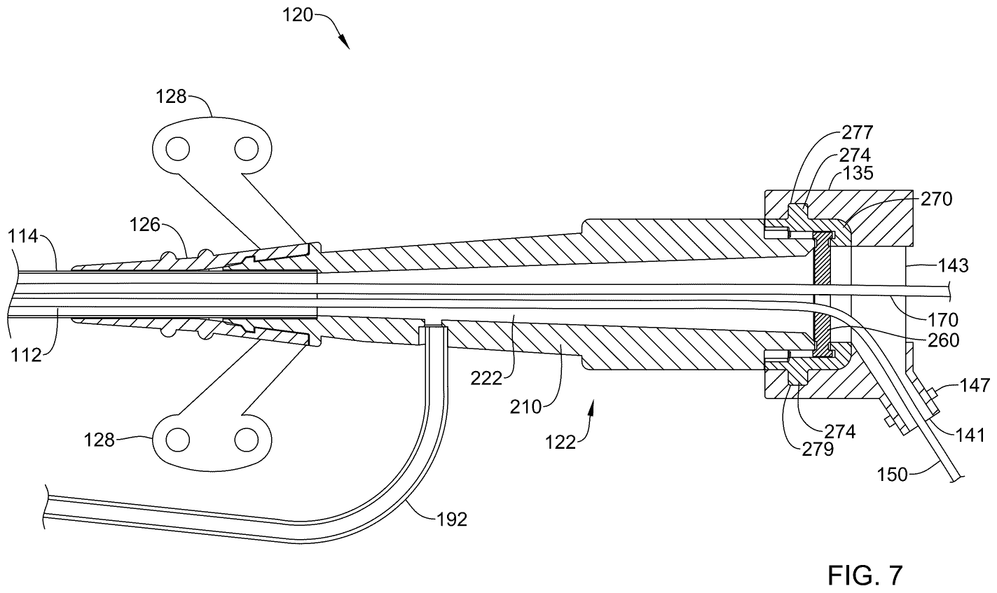

FIG. 7 is a cross-sectional view of the introducer sheath with a plurality of medical devices passing through the port 122, including through the elastomeric seal 260.

In some instances, the port 122 may be configured to accommodate a medical device having a diameter of 12 French (F) or more, 14 F or more, 15 F or more, 16 F or more, or 17 F or more. In other words, any seal(s) such as the elastomeric seal 260 may be configured to permit a medical device having a diameter of 12 F or more, 14 F or more, 15 F or more, 16 F or more, or 17 F or more, to pass therethrough while maintaining hemostasis. For instance, the port 122 may be configured to receive a plurality of medical devices at the same time with at least one of the medical devices (e.g., a blood pump) having a diameter of 12 F or more, 14 F or more, 15 F or more, 16 F or more, or 17 F or more. In other words, the elastomeric seal 260 may be configured to permit a medical device having a diameter of 12 F or more, 14 F or more, 15 F or more, 16 F or more, or 17 F or more, to pass therethrough while maintaining hemostasis.

In use, an elongate shaft of a plurality of medical devices 150, 170 may be inserted via an access port (e.g., access port 141 and/or 143) through the port introducer 135 and through the elastomeric seal 260 and the passageway 222 of the port 122 and remain inserted in the passageway 222 of the port 122 at the same time. For instance, the elongate shafts of the plurality of medical devices 150, 170 may be inserted via separate access ports of the port introducer 135 through the elastomeric seal 260, through the passageway 222 of the port 122, and through the lumen 112 of the elongate shaft 114 at the same time, as illustrated in FIG. 7.

For example, the elongate shafts of the medical devices 150, 170 may be passed through separate access ports of the longitudinal port introducer 135 and through the common passageway 224 of the longitudinal port introducer 135. For instance, a first medical device 150 (e.g., blood pump 150) may be passed through and remain in the first access port 141 while a second medical device 170 (e.g., a catheter, guidewire, etc.) may be passed through and remain in the second access port 143 as illustrated in FIG. 7, with both elongate shafts of the medical devices 150, 170 passing through the common passageway 224 of the longitudinal port introducer 135. In some instances, the first medical device may be a blood pump and the second medical device may be a different type of medical device such as a catheter, a guidewire, a stent, etc. As illustrated in FIG. 7, the first access port 141 of the longitudinal port introducer 135 may extend at an angle relative to the second access port 143. For instance, a planar face of a first opening opening into the first access port 141 may be at the acute angle relative to a planar face of a second opening opening into the second access port 143.

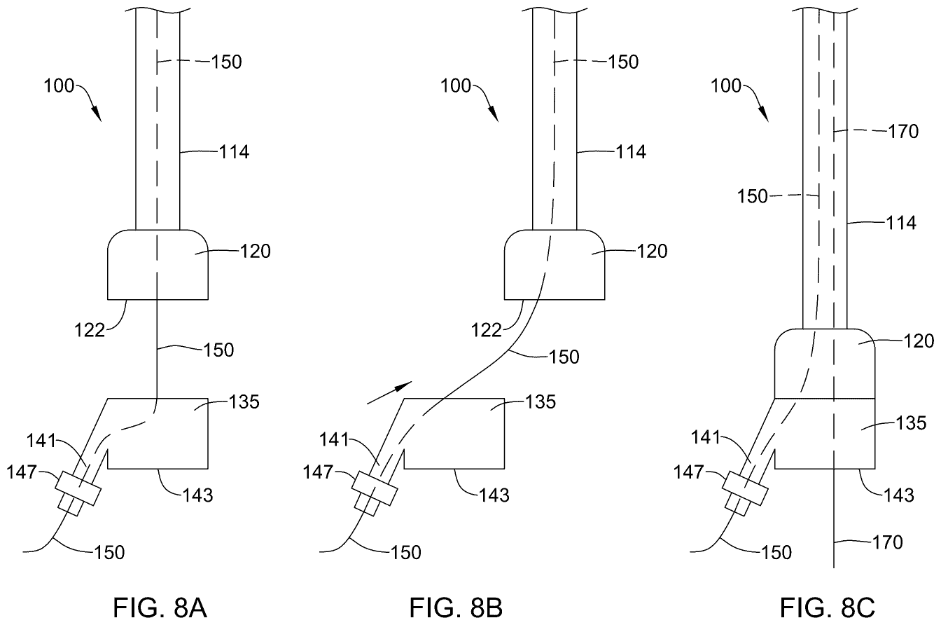

As mentioned herein, having the first access port 141 be at an angle (e.g., an acute angle) relative to the second access port 143 may promote aspects herein. Some aspects of inserting and advancing multiple medical devices through the introducer sheath 100 are illustrated in FIGS. 8A-8C. For instance, as shown in FIG. 8A, a first medical device 150 such as a blood pump (e.g., blood pump 150) may be inserted in the port 122 of the hub 120 of the introducer sheath 100 in an axial direction parallel to the longitudinal axis of the elongate shaft 114 of the introducer sheath 100. In other words, the elongate shaft of the first medical device 150 may be inserted into the introducer sheath 100 through the port 122 of the hub 120 with the elongate shaft of the first medical device 150 aligned with the longitudinal axis of the elongate shaft 114 of the introducer sheath 100. In some instances, the longitudinal port introducer 135 may be preloaded on the elongate shaft of the first medical device 150 prior to inserting the first medical device into the port 122 of the hub 120 of the introducer sheath 100, with the longitudinal port introducer 135 disconnected or decoupled from (e.g., not attached to) the hub 120 of the introducer sheath 100. For instance, the elongate shaft of the first medical device 150 may extend through the passage 224 of the longitudinal port introducer 135 such that a distal end region of the first medical device 150 extends distal of the longitudinal port introducer 135 and a proximal end region of the first medical device 150 extends out the first access port 141 and extends proximal of the longitudinal port introducer 135.

Once the elongate shaft of the first medical device 150 has been inserted into the hub 120 of the introducer sheath 100 and advanced into or through the elongate shaft 114 of the introducer sheath 100, the longitudinal port introducer 135 can be advanced distally along the elongate shaft of the first medical device 150 to couple the longitudinal port introducer 135 to the proximal end of the port 122 of the hub 120 of the introducer sheath 100, as shown in FIG. 8B. In advancing the longitudinal port introducer 135 toward the port 122 of the hub 120 of the introducer sheath 100 and coupling the longitudinal port introducer 135 thereto, the portion of the elongate shaft of the first medical device 150 extending proximally from the port 122 may be moved away from the longitudinal axis of the elongate shaft 114 of the introducer sheath 100.

With the longitudinal port introducer 135 coupled to the port 122 of the hub 120 of the introducer sheath 100, the longitudinal port introducer 135 may redirect the portion of the elongate shaft of the first medical device 150 extending proximally from the hub 120 away from the longitudinal axis of the elongate shaft 114 of the introducer sheath 100, providing in-line access for the second medical device 170 to be inserted into the second port 143 of the longitudinal port introducer 135, through the port 122 of the hub 120 and into the elongate shaft 114 of the introducer sheath 100 while substantially parallel to the longitudinal axis of the elongate shaft 114 of the introducer sheath 100. Once the first medical device 150 has been advanced distally through the introducer sheath 100 to position the first medical device 150 at a desired position in the patient's body, the tightening port 147 of the first access port 141 may be actuated to secure the first medical device 150 from further movement relative to the introducer sheath 100. In other words, the first medical device 150 may be axially restrained from further movement with the tightening port 147.

With the longitudinal port introducer 135 coupled to the hub 120, the second access port 143 may be axially aligned with the longitudinal axis of the elongate shaft 114 of the introducer sheath 100. In other words, the longitudinal axis of the elongate shaft 114 of the introducer sheath 100 may extend through the second access port 143 such that a medical device subsequently inserted into the introducer sheath 100 through the second access port 143 may be aligned with the longitudinal axis and advanced distally in-line with the longitudinal axis of the introducer sheath 100.

With the longitudinal port introducer 135 coupled to the hub 120 and the first medical device 150 moved away from the longitudinal axis of the introducer sheath 100, the second medical device 170 may be inserted into the second access port 143 and advanced into the introducer sheath 100 through the port 122 of the hub 120, as shown in FIG. 8C. Thus, each of the first medical device 150 and the second medical device 170 may be sequentially advanced into the port 122 of the hub 120 of the introducer sheath 100 in an axial direction (i.e., advanced distally through the port 122 of the hub 120 of the introducer sheath along the longitudinal axis of the elongate shaft 114 of the introducer sheath 100).

The first medical device 150 may extend through the first access port 141, through the port 122 of the hub 120 of the introducer sheath 100, and into the lumen of the elongate shat 114 of the introducer sheath 100 while the second medical device 170 is inserted into the second access port 143, through the port 122 of the hub 120 of the introducer sheath 100, and into the lumen of the elongate shaft 114 of the introducer sheath 100 at least due to the presence of the angle which promotes the first medical device 155 to remain disposed principally along a given side surface of the longitudinal port introducer 135 and/or of the introducer sheath 100 thereby ensure there is sufficient clearance for the insertion and viable functioning of the second medical device 170 along the longitudinal axis of the longitudinal port introducer 135 and/or the elongate shaft 114 of the introducer sheath 100.

Returning to FIG. 7, the elongate shafts of the medical devices 150, 170 may each be passed through an opening of the elastomeric seal 260 when inserted into the port 122 of the introducer sheath 100. When the proximal portion of the elongate shaft of the first medical device 150 is moved away from the longitudinal axis of the elongate shaft 114 of the introducer sheath 100 to provide in-line advancement (e.g., advancement of the second medical device 170 along the longitudinal axis of the elongate shaft 114 of the introducer sheath 100) the deflection of the first medical device 150 away from the longitudinal axis may cause the elastomeric seal 260 to flex and/or deform to permit the elongate shaft of the second medical device 170 to be advanced therethrough along the longitudinal axis of the elongate shaft 114 of the introducer sheath 100. The elastomeric seal 260 may seal around an outer perimeter of the elongate shaft of the medical device 150 and substantially prevent blood from escaping from the port 122. Thereafter, the elongate shaft of the second medical device 170 may pass through the elastomeric seal 260 into the passageway 222 of the hub 120, and may be further advanced through the elongate shaft 114 of the sheath 100 into the body of the patient for a diagnostic and/or therapeutic procedure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An introducer sheath assembly, comprising:
an introducer sheath including:
a hub at a proximal end region of the introducer sheath, the hub defining a port; and
an elongate shaft extending distally from the hub to a distal end region of the introducer sheath, the elongate shaft defining a lumen extending therethrough; and
a longitudinal port introducer extending proximally from the port of the hub, the longitudinal port introducer including:
a housing defining a passageway extending therethrough; and
a plurality of access ports in the housing, the plurality of access ports including a first access port and a second access port, wherein the first access port extends at an oblique angle from the second access port.

2. The introducer sheath assembly of claim 1, wherein the longitudinal port introducer is integral with the hub.

3. The introducer sheath assembly of claim 1, wherein the longitudinal port introducer is removably coupled to the hub.

4. The introducer sheath assembly of claim 1, wherein the angle is an acute angle.

5. The introducer sheath assembly of claim 1, wherein the first access port extends through a side surface between the proximal end and the distal end of the longitudinal port introducer.

6. The introducer sheath assembly of claim 1, wherein the first access port, the second access port, or both, further comprises a tightening port.

7. The introducer sheath assembly of claim 1, wherein the second access port extends through a proximal end of the housing of the longitudinal port introducer.

8. The introducer sheath assembly of claim 1, wherein the plurality of access ports are each configured to receive a separate medical device and provide the respective medical device access to the lumen of the elongate shaft of the introducer sheath.

9. The introducer sheath assembly of claim 1, wherein at least one of the access ports in the longitudinal port introducer includes an elastomeric seal.

10. The introducer sheath assembly of claim 9, wherein the elastomeric seal is configured to seal the second access port.

11. The introducer sheath assembly of claim 10, wherein the elastomeric seal is included in a plurality of elastomeric seals that include a first elastomeric seal and a second elastomeric seal, wherein the housing further comprises a third access port, and wherein the first elastomeric seal is configured to seal the second access port and the second elastomeric seal is configured to seal the third access port.

12. The introducer sheath assembly of claim 1, wherein the hub includes a hemostasis valve.

13. The introducer sheath assembly of claim 12, wherein the longitudinal port introducer includes a seal displacement mechanism.

14. The introducer sheath assembly of claim 13, wherein the seal displacement mechanism includes one or more protrusions extending distally from the housing of the longitudinal port introducer configured to engage the hemostasis valve.

15. The introducer sheath assembly of claim 14, wherein the one or more protrusions is configured to open the hemostasis valve when the longitudinal port introducer is coupled to the hub.

16. An introducer sheath assembly, comprising:
an introducer sheath including a hub at a proximal end of the introducer sheath, the hub defining a port including a hemostasis valve therein;
an elongate shaft extending distally from the hub to a distal end of the introducer sheath, the elongate shaft defining a lumen extending therethrough; and
a longitudinal port introducer detachably coupled to the port of the hub, the longitudinal port introducer including:
a housing defining a passageway extending therethrough; and
a plurality of access ports in the housing, the plurality of access ports including a first access port and a second access port, wherein the second access port is axially aligned with the port of the hub and the first access port extends at an angle from the second access port, and wherein each of the first and second access ports is configured to receive a separate medical device therethrough and provide the respective medical device access to the port of the hub for advancement into the lumen of the elongate shaft of the introducer sheath.

17. The introducer sheath assembly of claim 16, wherein the longitudinal port introducer includes a seal displacement mechanism extending distally from the housing of the longitudinal port introducer, wherein the seal disengagement mechanism is configured to engage the hemostasis valve when the longitudinal port introducer is coupled to the port of the hub.

18. A method of advancing a plurality of medical devices through an introducer sheath, comprising:
advancing a first medical device through a port of a hub of the introducer sheath in an axial direction extending along a longitudinal axis of the introducer sheath; and
advancing a second medical device through a second access port of a longitudinal port introducer coupled to the hub of the introducer sheath and through the port of the hub of the introducer sheath in an axial direction extending along the longitudinal axis of the introducer sheath while a proximal end region of the first medical device extends proximally through a first access port of the longitudinal port introducer at an angle to the second access port.

19. The method of claim 18, further comprising:
coupling the longitudinal port introducer to the hub of the introducer sheath after advancing the first medical device through the port of the hub of the introducer sheath.

20. The method of claim 19, wherein the longitudinal port introducer is pre-loaded onto the first medical device prior to advancing the first medical device through the port of the hub of the introducer sheath.
